(19) United States
(12) Patent Application Publication
(10) Pub. No.: US 2022/0297119 A1
(43) Pub. Date: Sep. 22, 2022

(54) COMPOUND INTRODUCTION APPARATUS AND COMPOUND INTRODUCTION METHOD

(71) Applicant: CANON KABUSHIKI KAISHA, Tokyo (JP)

(72) Inventors: Shinichi Sakurada, Tokyo (JP); Sachiko Yamauchi, Kanagawa (JP); Tatsuaki Orihara, Tokyo (JP); Tsutomu Shiratori, Tokyo (JP); Nobuyuki Kuwabara, Tokyo (JP); Ryosuke Hirokawa, Kanagawa (JP); Yoshinori Itoh, Kanagawa (JP); Futoshi Hirose, Kanagawa (JP); Takeshi Okada, Kanagawa (JP)

(21) Appl. No.: 17/693,836

(22) Filed: Mar. 14, 2022

(30) Foreign Application Priority Data
Mar. 18, 2021 (JP) 2021-044714
Mar. 10, 2022 (JP) 2022-037194

(51) Int. Cl. B01L 3/00; C12N 15/87

(52) U.S. Cl. CPC B01L 3/502715; C12N 15/87; B01L 2200/027; B01L 2200/0647; B01L 2200/16; B01L 2300/1827; B01L 2400/0442

(57) ABSTRACT

One embodiment of the present invention provides a compound introduction apparatus for introducing a compound into a cell, including: a supply flow channel for supplying a cell suspension containing the cell and the compound; an introduction unit configured to introduce the compound into the cell; and an ejection port for ejecting the cell suspension containing the cell in which the compound has been introduced. A height of the channel relative to a flow direction of the cell suspension through the channel is more than 1.0 times a diameter of the cell and less than 2.0 times the diameter, and a width of the channel relative to the flow direction is more than 1.0 times the diameter of the cell and less than 2.0 times the diameter, and a diameter of the ejection port is more than 1.0 times the diameter of the cell and less than 2.0 times the diameter.

### BACKGROUND

### Field

The present disclosure relates to a compound introduction apparatus and compound introduction method for introducing a compound into a cell.

### Description of the Related Art

Techniques for introducing a compound including a gene into a cell have been known. The introduction method includes a chemical or biological introduction method using a cationic substance or a virus on a living cell. Also, various methods have been developed including physical introduction methods such as an electroporation method and a gene gun method, which are expected to be low in toxicity, and a microinjection method, with which a wide range of compounds to be introduced are selectable and introduction is reliable.

In recent years, due to the emergence of cell therapeutic drugs and induced pluripotent stem cells, which are obtained by introducing compounds including genes into cells to thereby modify the cells' properties, there has been a demand for a more efficient compound introduction method. However, means for achieving such efficiency have not been established, which has been a problem.

As a means for solving the above problem, the specification of U.S. Patent Application Publication No. 2018/0003696 discloses an introduction method using a micro flow channel. In this method, cells dispersed in a liquid containing a compound to be introduced are passed through a micro flow channel having substantially the same size as the cells. In this way, shear force is applied to the cells, so that holes are temporarily formed in their cell membranes. Thus, the compound to be introduced outside the cells can be introduced into the cells, and therefore the compound can be efficiently introduced into each cell.

The description of Japanese Patent No. 5645657 discloses that, by using an inkjet device for use as an image printing apparatus to generate pressure and shear force inside a micro-sized space therein, a compound of interest can be effectively introduced into cells.

### SUMMARY

An object of one embodiment of the present invention is to achieve both efficient introduction of a compound into a cell and efficient generation of a cell whose properties are modified by the introduction.

A compound introduction apparatus according to an aspect of the present invention is a compound introduction apparatus for introducing a compound into a cell, including: a supply flow channel for supplying a cell suspension containing the cell and the compound; an introduction unit configured to introduce the compound into the cell; and an ejection port for ejecting the cell suspension containing the cell in which the compound has been introduced, in which a height of the supply flow channel relative to a flow direction of the cell suspension flowing through the supply flow channel is more than 1.0 times a diameter of the cell and less than 2.0 times the diameter of the cell, and a width of the supply flow channel relative to the flow direction is more than 1.0 times the diameter of the cell and less than 2.0 times the diameter of the cell, and a diameter of the ejection port is more than 1.0 times the diameter of the cell and less than 2.0 times the diameter of the cell.

Further features of the present invention will become apparent from the following description of exemplary embodiments with reference to the attached drawings.

### BRIEF DESCRIPTION OF THE DRAWINGS

FIG. 1 is a diagram illustrating one example of a compound introduction apparatus;

FIG. 2 is a block diagram illustrating one example of a control system of the compound introduction apparatus;

FIG. 3 is a perspective view illustrating an exterior of an ejection head cartridge;

FIGS. 4A to 4C are views illustrating an example of the positional relationship among components formed on an electronic substrate; and

FIG. 5 is a view schematically illustrating a state of a cell suspension in a flow channel in an ejection head.

### DESCRIPTION OF THE EMBODIMENTS

According to the present inventors' consideration, in a case of employing the method disclosed in the specification of U.S. Patent Application Publication No. 2018/0003696, it is necessary to prepare a micro flow channel slightly narrower than the cell size for each cell type. Moreover, in the case of sending a liquid into the micro flow channel, the pressure drop is so large that an extremely large force is required. Furthermore, it has been found that clogging of the flow channel with a cell occurs frequently, which is a problem in view of the production efficiency of the introduction process.

It has also been found that the method disclosed in the description of Japanese Patent No. 5645657 cannot achieve efficient compound introduction either.

As a result, the present inventors have made vigorous consideration to provide a compound introduction apparatus capable of achieving both efficient introduction of a compound into a cell and efficient generation of a cell whose properties are modified by the introduction, and have arrived at the present invention.

Embodiments according to the present invention will be described below with reference to the drawings. Note that the following embodiments do not limit the present invention, and not all the combinations of the features described in these embodiments are necessarily essential. Incidentally, the same components will be described with the same reference sign given thereto.

### First Embodiment

### <Compound Introduction Apparatus>

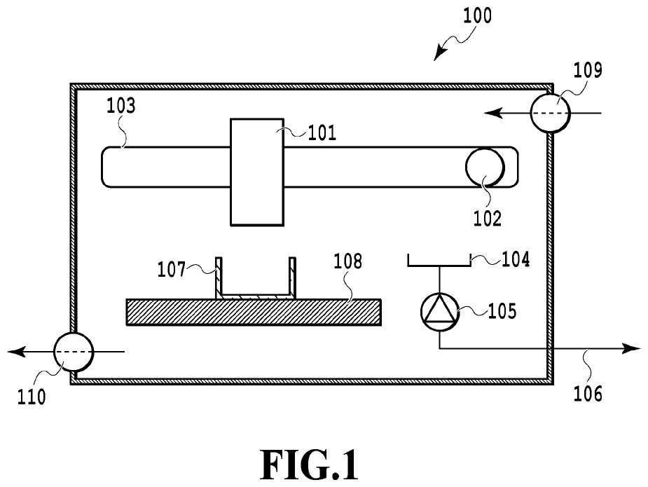

FIG. 1 is a diagram illustrating one example of a compound introduction apparatus 100 in the present embodiment. The compound introduction apparatus 100 in the present embodiment is an apparatus including an inkjet-type ejection head 101. The ejection head 101 is filled with a liquid containing a compound and cells into which the compound is to be introduced. Herein, this liquid will be referred to as “cell suspension” (also called “cell-containing liquid”). The ejection head 101 may be called “cell processing head” or “liquid ejection head”. The cell suspension after being ejected from the ejection head 101 contains cells into which the compound has been introduced. The compound introduction apparatus 100 as above may be called “ejection apparatus” or “cell processing apparatus”. A configuration of the compound introduction apparatus 100 will be described below.

FIG. 1 is a diagram schematically illustrating the compound introduction apparatus 100 in the present embodiment. The compound introduction apparatus 100 illustrated in FIG. 1 includes a driving motor 102, a driving belt 103 connected to the driving motor 102, and the ejection head 101 to be conveyed by the driving belt 103. The ejection head 101 can be moved to any position within a region within which the ejection head 101 can be conveyed, by driving the driving motor 102.

The liquid filled in the ejection head 101 is filled into flow channels and ejection ports in the ejection head 101 by bringing the ejection head 101 into contact with a suction mechanism 104 and actuating a suction motor 105. Also, in a case where ejection has not been performed for a certain period of time, the liquid inside the ejection head 101 can be discharged by bringing the ejection head 101 into contact with the suction mechanism 104 and actuating the suction motor 105. The liquid resulting from the suction and the discharge is discharged to the outside of the apparatus through a waste liquid tube 106. Incidentally, the suction mechanism 104 can also be used as a cap to prevent the head from becoming dry while no introduction process is performed.

The cells with the compound introduced therein are ejected from the ejection head 101 toward a culture dish 107 fixed to the top of a processing stage 108. The cells with the compound introduced therein can be obtained in this manner.

Mist and dust inside the apparatus can be reduced by an air suction port 109 having a fan and a filter and an air discharge port 110. This makes it possible to introduce the compound while also reducing contamination.

### <Control Block Diagram>

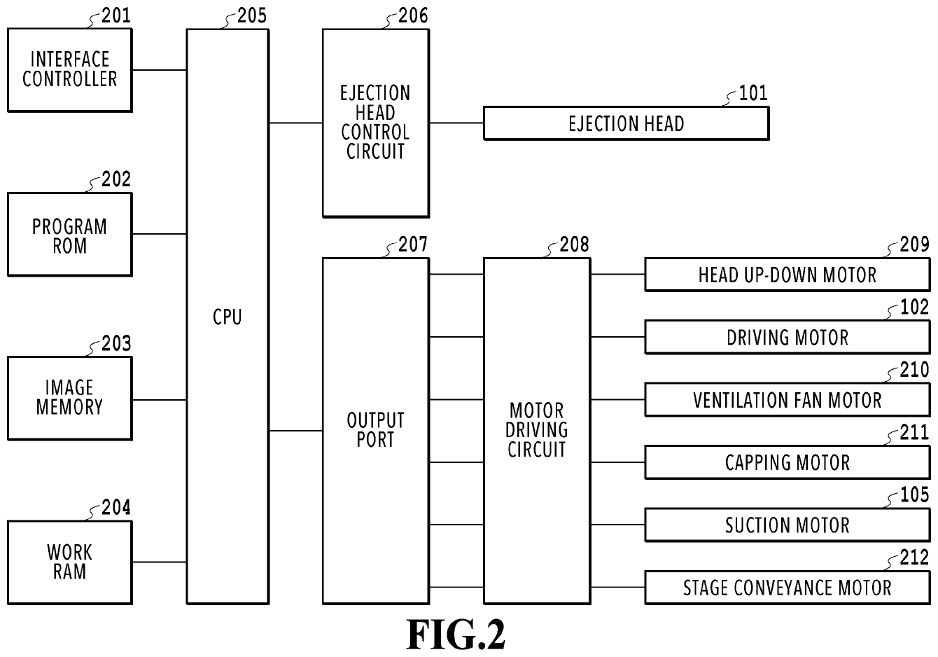

FIG. 2 is a block diagram illustrating one example of a control system included in the compound introduction apparatus 100 in the present embodiment. The compound introduction apparatus 100 has the CPU 205, an interface controller 201, a program ROM 202, an image memory 203, a work RAM 204, an ejection head control circuit 206, an output port 207, and a motor driving circuit 208. The present embodiment will be described on the assumption that the compound introduction apparatus 100 controls introduction of any of various compounds in response to an instruction from a host apparatus, such as an external apparatus. However, the present embodiment is not limited to this case. The compound introduction apparatus 100 may be provided with an operation unit or the like and control introduction of any of various compounds in response to an instruction corresponding to an operation on the operation unit.

Output data and a command are sent to the compound introduction apparatus 100 from a host information terminal (e.g., a personal computer, a smartphone, a tablet device, or the like). The CPU 205, which is a control unit, receives these output data and command via the interface controller 201. The CPU 205 is an arithmetic processing unit that performs control of the entire compound introduction apparatus 100, such as receiving the output data, performing an introduction operation, conveying the ejection head, and so on. The CPU 205 analyzes the received command and, thereafter, loads and stores control signal data in the image memory 203, the control signal data being data for actuating the ejection head 101.

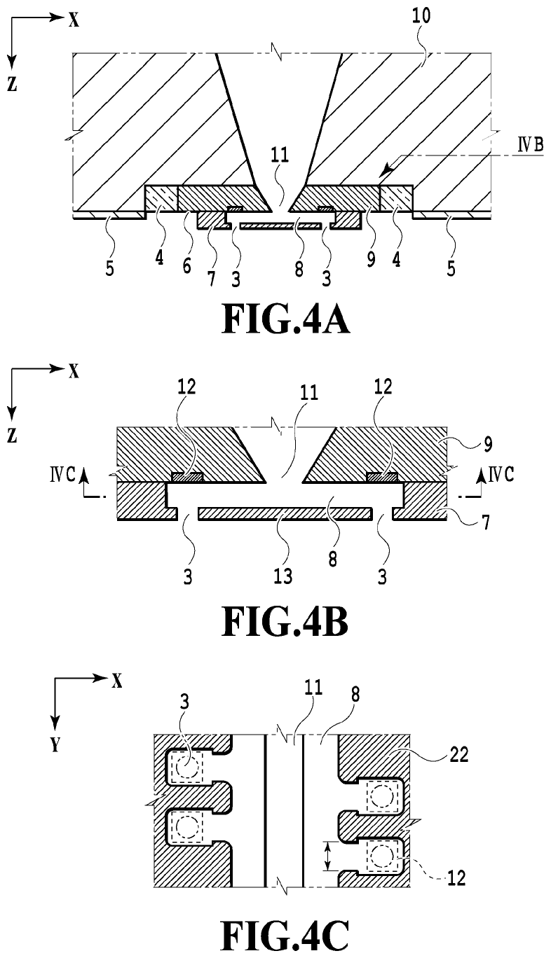

As an operation process to be performed before the compound introduction, the CPU 205 reads out the control signal data to be used in the introduction control from the image memory 203 in synchronization with conveyance of the ejection head 101. Then, the CPU 205 transfers this read data to the ejection head 101 via the ejection head control circuit 206. Further, the CPU 205 causes the ejection head 101 to process cells therein as needed to introduce the compound into them. Thereafter, the CPU 205 causes the ejection head 101 to eject the cell suspension from its ejection ports 3 (see FIG. 4A, etc.) toward the culture dish 107 from directly above the culture dish 107. Note that the control signal data may be stored in the work RAM 204, which is a work memory, instead of the image memory 203.

The operation of the CPU 205 is executed based on a processing program stored in the program ROM 202. The program ROM 202 stores the processing program, tables, and the like for the control process. The work RAM 204 stores the time elapsed since the end of the last introduction process, and the CPU 205 executes commands of a capping process, an ejection port suction process, and the like according to the time elapsed. Also, the CPU 205 uses the work RAM 204 as a work memory.

The motor driving circuit 208 drives various motors such as a head up-down motor 209, the driving motor 102, a ventilation fan motor 210, a capping motor 211, the suction motor 105, and a stage conveyance motor 212.

Components forming the compound introduction apparatus 100 in the present embodiment will be described below in detail.

### <Ejection Head>

The compound introduction apparatus 100 in the present embodiment can introduce the compound into cells by instantaneously applying at least mechanical energy to the cell suspension by using energy generation elements each of which is an introduction unit configured to introduce the compound into a cell. As a method that enables instantaneous energy application as above, a piezoelectric inkjet method can be used in which a piezoelectric element is disposed in a micro flow channel and exhibits a mechanical action by using its displacement caused via voltage application. Alternatively, a thermal inkjet method can be used in which a heater (heating element) having high electrical resistance is disposed in a flow channel, and the heater is caused to generate heat via voltage application to thereby generate a bubble on the order of microseconds in a liquid near the heater to exhibit a mechanical action and a thermal action. Using the thermal inkjet method is preferable since mechanical energy can be applied in an extremely small space and actuator units can be arranged densely. Thus, each energy generation element is preferably a heat generation element. It is also preferable that, in a case where the heat generation element generates heat, a stress resulting from bubble generation caused by the heat generation be applied to the cell suspension inside a processing chamber. Note that the heat generation time of the heater is preferably 0.05 μs or more and 10 μs or less since in this way a bubble generation phenomenon can occur, and more preferably 0.1 μs or more and 5 μs or less since in this way cell death can be inhibited.

Also, it is preferable that the heat generation time of the heater be 1 μs or more and 10 μs or less since in this way a stress is exerted on the cell suspension for a span of 1 μs or more and 10 μs or less and as a result a droplet is discharged from the ejection port at a speed of 0.5 m/s or more and 30 m/s or less.

The thermal action of the heater brings about an effect of promoting expression of a heat shock protein gene in the cell or a gene that promotes cell division, as described by Campbell et al. in “Campbell, A et al., 2020 Frontiers in Bioengineering and Biotechnology, 8 doi: 10.3389/fbioe.2020.00082”. In view of efficiently introducing the compound into dividing cells, the thermal inkjet method is preferable.

Also, each energy generation element is preferably one that generates energy in the height direction of a supply flow channel. In this way, it is possible to efficiently form holes in the surface membrane of a cell.

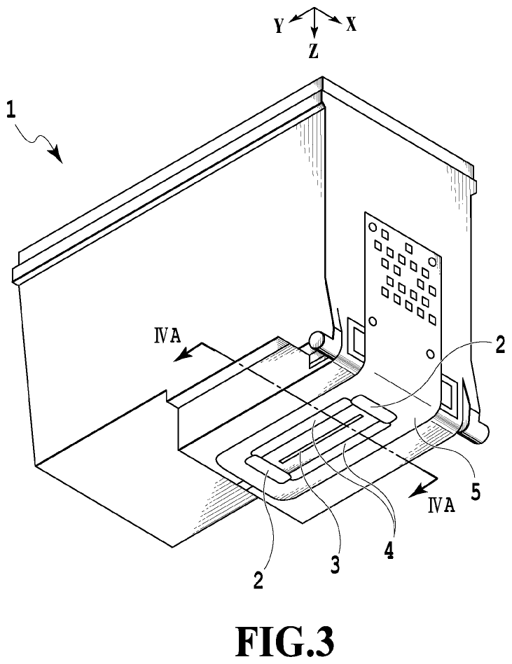

A configuration of the ejection head 101 in the present embodiment will be described below using FIGS. 3 and 4A to 4C. FIG. 3 is a perspective view illustrating a configuration of a cartridge of the ejection head 101 (hereinafter referred to as “ejection head cartridge”). The ejection head cartridge 1 includes a liquid tank part as a liquid supply source (referred to also as “cell suspension storage part”), and an ejection head in the form of a chip provided integrally with the liquid tank part. The ejection head is attached to a liquid supply-hold part 10 (see FIGS. 4A to 4C) with a second sealant 4. Also, an electrical wiring member 5 for electrically connecting the ejection head and the body side is arranged from the liquid supply-hold part 10 to the liquid tank part. Connections (not illustrated) of the electrical wiring member 5 to the ejection head's electrodes are each sealed by a first sealant 2. Note that coordinates axes are set as illustrated in FIG. 3. These coordinates axes are used in the subsequent drawings too.

FIGS. 4A to 4C are views illustrating an example of the positional relationship among components formed on an electronic substrate included in the ejection head. FIG. 4A is a vertical cross-sectional view along line IVA in FIG. 3. FIG. 4B is an enlarged view of a part of FIG. 4A, specifically, the part indicated by line IVB in FIG. 4A. FIG. 4C is a horizontal cross-sectional view along line IVC in FIG. 4B.

As illustrated in FIGS. 4A to 4C, a silicon substrate 9 attached to the liquid supply-hold part 10 with the second sealant 4 is provided with a liquid supply port 11 for receiving a liquid supplied from the liquid tank and supplying the received liquid to the ejection ports 3 arranged in lines. As illustrated in FIGS. 4B and 4C, the liquid supply port 11 is formed to extend over the entire length of the ejection port arrays arranged in lines on both sides of the liquid supply port 11. The liquid supply port 11 receives the liquid supplied from the liquid supply source, and this liquid is supplied to each of the plurality of ejection ports in each of the plurality of ejection port arrays, which form an ejection part. As illustrated in FIG. 4C, the substrate 9 is provided with heat generation elements 12 for the respective ejection ports 3. Also, to the substrate 9 is joined an ejection port plate 7 in which are formed flow channel walls 22 separating liquid supply flow channels (referred to also as “supply flow channels”) 8 through which the liquid supply port 11 and the ejection ports 3 communicate with each other and which guide the liquid. The flow channel walls 22 are each formed of a flow channel forming member. The ejection ports 3 are arranged at predetermined intervals along the longitudinal direction of the liquid supply port 11 (the Y direction in the drawings).

As illustrated in FIGS. 4A to 4C, the ejection head has the substrate 9, the liquid supply port 11, the heat generation elements 12, the ejection ports 3, the ejection port plate 7, the liquid supply flow channels 8, through which the liquid supply port 11 and the ejection ports 3 communicate with each other, and the flow channel walls 22 separating the adjacent liquid supply flow channels 8.

A wiring pattern is formed on the substrate 9 in addition to the heat generation elements 12 (e.g., electrothermal transducers). Moreover, the ejection port plate 7, or a structure which is made of a resin material and in which the arrays of ejection ports 3 and the liquid supply flow channels 8 are formed, is formed by a photolithographic technique. A water repellent layer 13 is formed on the ejection port plate 7 except the portions of the ejection ports 3. Thus, this water repellent layer 13 forms the outermost surface of the ejection head. Moreover, electrical wirings, electrode portions 6, and the like are formed on the substrate 9.

The electrical wiring member 5 is a member for forming electrical signal channels for supplying electrical signals to electrode wirings on the substrate 9. In the electrical wiring member 5, an opening portion for installing the substrate 9 is formed. Electrode terminals (not illustrated) to be connected to the electrode portions 6 of the chip are formed near the edge of this opening portion. The chip and the electrical wiring member 5 are bonded to the liquid supply-hold part 10, which is formed by molding a resin. Electrical connections between the substrate 9 and the electrical wiring member 5 are sealed by the first sealant 2, which seals lower portions of the electrical connections and portions of the chip where the electrodes are not formed, and the second sealant 4, which seals upper portions of the electrical connections. The sealing by the first sealant 2 and the second sealant 4 protects the electrical connections from corrosion by the cell suspension and external impact.

### <Flow Channel Height and Flow Channel Width of Liquid Supply Portions>

The height, or the length in the Z direction (direction of gravity), and the width, or the length in the Y direction, of the liquid supply flow channels 8, which are liquid supply portions, are each more than 1.0 times the diameter of a cell and less than 2.0 times the diameter of a cell. Since the height and width of the liquid supply flow channels 8 are more than 1.0 times the diameter of a cell, the liquid supply flow channels 8 are unlikely to get clogged with cells. This makes it possible to supply the cell suspension containing the compound and cells to bubble generation portions in which the heat generation elements 12 are present (referred to also as “processing chambers”). Also, since the height of the liquid supply flow channels 8 is less than 2.0 times the diameter of a cell, when cells are supplied into the bubble generation portions, in which the heat generation elements 12 are present, the cells supplied are present near the heat generation elements 12. Thus, with such a configuration, it is possible to efficiently perform processing of forming holes in the surface membranes of cells to introduce the compound into the cells.

Note that the smallest height and width of the liquid supply flow channels are used as the height and width of the liquid supply flow channels mentioned above. For example, the width of each liquid supply flow channel in FIG. 4C means the length of the supply flow channel indicated by the arrows immediately before a bubble generation portion in the top of which a heat generation element 12 is present. Also, the shape of the liquid supply flow channels includes a straight shape, a meandering shape, and so on but is not limited to these shapes, and liquid supply flow channels of any shape may be employed. Also, the liquid supply flow channels may employ a structure that narrows in height and width toward the bubble generation portion from the liquid supply portion or employ a structure that maintains the same height and width toward the bubble generation portion from the liquid supply portion. Also, the height and width of the supply flow channels are dimensions relative to the flow direction of the cell suspension flowing through the supply flow channels.

Also, the height and width of the liquid supply flow channels may be selected as appropriate according to the diameter of the cells to be processed. Specifically, the height and width of the liquid supply flow channels are each preferably more than 1 μm and less than 200 μm.

### <Ejection Ports>

In the present embodiment, as illustrated in FIG. 4C, circular ejection ports 3 are employed but ejection ports of any shape may be selected as appropriate. Examples of the shape of the ejection ports include circular shapes, elliptical shapes, triangular shapes, rectangular shapes, square shapes, polygonal shapes, star shapes, shapes having a protrusion(s), and the like. However, the shape of the ejection ports is preferably such that the longest portion is at least 1.0 times longer than the shortest portion and at most 5.0 times longer than the shortest portion, and more preferably such that the longest portion is at least 1.0 times longer than the shortest portion and at most 2.0 times longer than the shortest portion.

Also, as the diameter of the ejection ports in the present embodiment, the length of their shortest portion is used. The diameter of the ejection ports is more than 1.0 times larger than the diameter of a cell and less than 2.0 times larger than the diameter of a cell. Since the diameter of the ejection ports is more than 1.0 times the diameter of a cell, the cell suspension receiving a stress as a result of bubble generation by the heat generation elements can be discharged without the flow channels being clogged with cells. Also, since the diameter of the ejection ports is less than 2.0 times the diameter of a cell, the compound is efficiently introduced by a stress received by the discharge.

As the ratio of the diameter of the ejection ports and the height of the liquid supply flow channels, any ratio can be employed. It is, however, preferable that the diameter of the ejection ports be larger than the height of the liquid supply flow channels. This is because, with the diameter of the ejection ports being larger than the height of the liquid supply flow channels, the liquid can be more easily discharged in the direction of the ejection ports. This reduces the variation in the positions of cells in the flow channels and hence enables stable processing.

### <Mechanism of Compound Introduction>

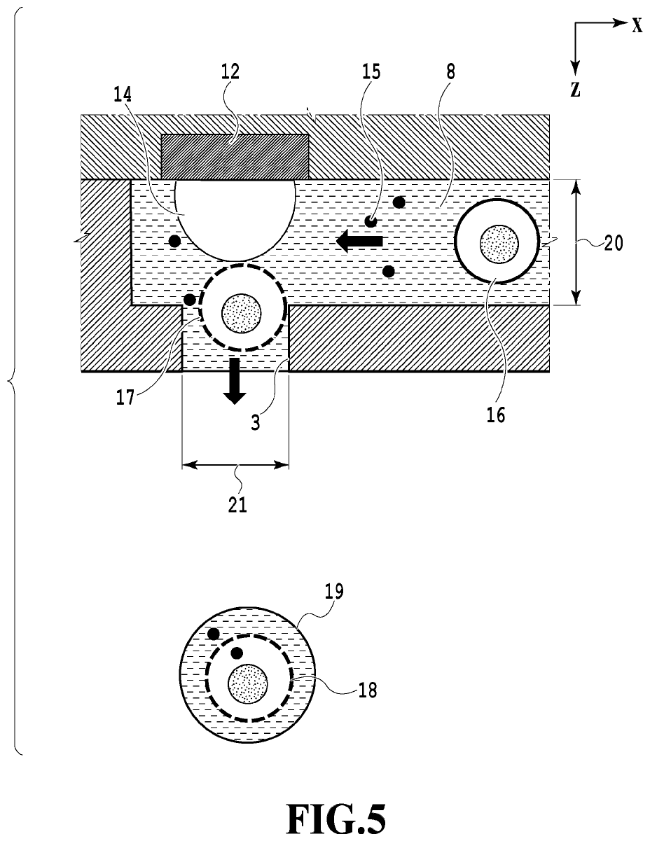

A presumed mechanism of the compound introduction by the compound introduction apparatus 100 in the present embodiment will be described below using FIGS. 4B and 5. FIG. 5 is an enlarged view of a part around a heat generation element 12 illustrated in FIG. 4B.

The cell suspension supplied from the liquid supply port 11 passes through the liquid supply flow channel 8 and is filled down to the ejection port 3. With the cell suspension thus filled, the heat generation element 12 is energized, so that the water in the cell suspension gets rapidly heated into a bubble generation state. As a result, a bubble is formed. Here, the cell suspension present at the position of a bubble 14 generated by the heating is rapidly pushed mainly in the direction of the ejection port 3 (+Z direction in FIG. 5), in which the flow resistance is small. At this time, a cell 16 yet to be processed contained in the cell suspension supplied through the liquid supply flow channel 8 turns into a cell 17 with holes formed in its cell membrane by a strong shear flow generated by the bubble generation. The cell in this state can let the compound, which normally cannot penetrate the cell membrane, temporarily penetrate the cell membrane through the holes formed therein. As a result, part of the compound contained in the cell suspension is taken into the cell. Also, the cell suspension pushed by the bubble generation is discharged through the ejection port 3, which is located directly below the heat generation element 12 and the bubble generation portion (the processing chamber in which the heat generation element is arranged). In order to facilitate the discharge of the cell suspension containing the cell in which the compound has been introduced, the ejection port is preferably arranged directly below the heat generation element and the processing chamber. In a case where the liquid discharged as a result of being pushed by the bubble generation has sufficient kinetic energy, this liquid is discharged in the form of a flying droplet. Also, the cell contained in the discharged cell suspension, in which the compound has been introduced, may be cultured under appropriate conditions. In this way, the temporarily formed holes in the cell membrane will close and, depending on the case, the cell will also be able to survive after that, thereby enabling continuous cell culture.

As described above, in the present embodiment, a height 20 and width of the liquid supply flow channels are each more than 1.0 times the diameter of a cell. In this way, the flow channels do not get clogged with cells, thereby enabling the cell suspension to be supplied to the bubble generation portions, in which the heat generation elements are present. Also, since the height 20 of the liquid supply flow channels is less than 2.0 times the diameter of a cell, when cells are supplied into the bubble generation portions, in which the heat generation elements 12 are present, the cells supplied are present near the heat generation elements 12. Thus, with a configuration as above, efficient processing is possible.

Also, a diameter 21 of the ejection ports is more than 1.0 times the diameter of a cell. In this way, the cell suspension receiving a stress as a result of bubble generation by the heat generation elements 12 can be discharged without the flow channels being clogged with cells. Further, the diameter 21 of the ejection ports is less than 2.0 times the diameter of a cell and is therefore not excessively large. Accordingly, the compound is efficiently introduced by a stress received at the time of discharge.

### <Compound Introduction Method>

A compound introduction method in the present embodiment has at least the following three steps.

These are: a step (first step) of supplying the cell suspension containing cells and the compound through the supply flow channels; a step (second step) of introducing the compound into the cells; and a step (third step) of ejecting the cell suspension containing the cells in which the compound has been introduced from the ejection ports. These steps will be described below by taking, as an example, a case where the introduction unit configured to introduce the compound is a heat generation element.

In the first step, the cell suspension containing the compound and cells into which the compound is to be introduced is passed through the liquid supply flow channels included in the liquid supply portions and caused to reach the bubble generation portions (processing chambers), in which the heat generation elements are present. Note that the height of these liquid supply flow channels is more than 1.0 times the diameter of a cell and less than 2.0 times the diameter of a cell, and the width of the liquid supply flow channels is more than 1.0 times the diameter of a cell and less than 2.0 times the diameter of a cell. Incidentally, the cell suspension may be introduced into the ejection head 101 from the cell suspension storage part of the ejection head cartridge 1 or directly introduced into the ejection head 101 with a micropipette or the like. In a case where the cell suspension is smoothly filled to the ejection ports of the ejection head 101 with wetting and spreading of the cell suspension due to surface tension, the introduction operation is executed as soon as the cell suspension is filled. In a case where the cell suspension cannot be filled to the ejection ports of the ejection head 101, the cell suspension can be filled via suction from the ejection ports with the suction mechanism 104 or an external suction pump. Alternatively, the cell suspension can be filled by pressurizing the reservoir part holding the cell suspension therein with an external pressurization pump.

In the second step, at the bubble generation portions, in which the heat generation elements are present, the heat generation elements are caused to generate heat. As a result of the second step, bubbles are generated in the cell suspension. Due to a stress generated by the bubble generation, a stress is applied to the cell suspension. This can introduce the compound into cells.

In the third step, the cell suspension containing the cells in which the compound has been introduced is ejected from the ejection ports. Note that the diameter of the ejection port is more than 1.0 times the diameter of a cell and less than 2.0 times the diameter of a cell. The cell suspension to be ejected is ejected to a base material or culture solution. The base material needs to be selected with the total load to be received by the cells taken into account, and is selected according to the purpose. Specifically, in a case of performing introduction processing by ejection onto a base material, it is preferable to immerse the cells in a culture medium containing a serum or an extracellular matrix or the like in advance and eject the cell suspension thus processed if the base material is an adherent base material whose surface has high affinity to the cells, such as glass or polystyrene. Also, in a case of using a fluororesin having low affinity to the cells, such as PTFE, or the like as the base material, it is preferable to transfer the ejection-target liquid into a culture medium after it is ejected. In a case of executing an introduction operation on a culture dish, it is preferable to bring the cells into contact with a culture medium containing a serum or an extracellular matrix on the culture dish in advance. It is also possible to directly eject the cells from the ejection head 101 onto a culture medium filled in a culture dish. The method is selected according to the purpose.

While the cell suspension can be transferred to a dish containing a culture medium that is optimal for the cells immediately after the ejection, the cell suspension can also be kept under a saturated aqueous vapor environment. In a case of increasing the amount to be introduced, it is preferable to let the cell suspension stand.

In the case of ejecting the cells onto a base material, a method involving removing the cell culture solution on the base material with a pipette or the like and seeding the cells onto a dish may be employed. In this case, it is possible to put the whole base material in the dish, add a culture medium, and culture the cells therein. Further, once the cell suspension is ejected, it may be introduced into the ejection head 101 and ejected therefrom again. In this way, a compound introduction operation can be repeated. In this case, it is preferable to use a base material whose surface has low affinity to the cells.

In the following description, the compound to be introduced into cells will also be referred to as “introduction-target compound”.

### <Diameter of Cells>

The diameter of the cells in the cell suspension is figured out by setting the cell suspension immediately before the processing on a hemacytometer or the like and measuring it with an optical microscope or the like equipped with an image sensor. By using an image recorded using the image sensor, the diameter of the cells can be figured out based distance information corresponding to images stored in advance. It is preferable to get the cells in focus so as to maximize their diameters, and then to record an image and measure the lengths.

### <Introduction-Target Compound>

The compound to be introduced can be selected as appropriate according to its purpose. Conceivable examples of introducible compounds include nucleic acids, proteins, labeling substances, and the like. Note that the compound is not limited to these examples as long as it is a compound of such a size as to be containable within a cell into which it is to be introduced. However, in view of minimizing damage to the cells, the size of the compound is preferably ⅕ of the average diameter of the cells or smaller, and more preferably 1/10 of the average diameter of the cells or smaller.

The size of the cells can be measured by processing the cells with an enzyme or the like and then analyzing a cell suspension thus obtained with a laser diffraction particle size analyzer or the like. Alternatively, the size of the cells can be measured by placing the cell suspension on a hemacytometer or the like and observing it with an optical microscope. Moreover, the size of the compound to be introduced can also be measured in a similar manner. In particular, for a compound of a size of 1 μm or smaller, a dynamic light scattering particle size analyzer can be used. Representative compounds that can be employed in the present embodiment include nucleic acids.

### <Nucleic Acids>

For the purpose of transient and stable expression of a nucleic acid or interference with a gene, an exogenous ribonucleic acid (RNA) or deoxyribonucleic acid (DNA) not different from ones derived from the compound introduction-target cells can be used as a compound to be introduced. As a nucleic acid higher-order structure, a single-stranded primary structure or a secondary structure, such as a hairpin-shaped stem-loop structure or a helix structure, can be used. Also, a tertiary structure, such as A-form, B-form, or Z-form, can be used. Also, a quaternary structure, such as a supercoiled shape, can be used. These higher-order structures can be preferably used according to the purpose. Also, these nucleic acids may be labeled with a fluorescent compound or a radioisotope, and any of these may be used according to the purpose.

### <RNA>

RNAs to be handled in the present embodiment include messenger RNA, which is responsible for copying and carrying a sequence from DNA to the ribosome, which is a site where protein synthesis takes place. The RNAs also include ribosome RNA, which is a substance forming the ribosome, and transfer RNA, which carries amino acids of corresponding sequences to the ribosome. Others include small nuclear RNA, small nucleolar RNA, microRNA, and siRNA, which exhibits an interfering action, and the like. However, the RNAs are not limited to these, and a preferable RNA can be used according to the purpose.

### <DNA>

As a DNA to be handled in the present embodiment, any of a single-stranded DNA, a double-stranded DNA, a triple-stranded DNA, and a four-stranded DNA can be selected. As its shape, a linear shape, a circular shape, or the like is generally used. In recent years, however, the shape is not limited and any shape can be used, as represented by DNA origami. A double-stranded DNA is preferable in view of substance stability, and a circular plasmid DNA is more preferable in view of ease of culture with Escherichia coli or yeast. Further, to be introduced into a cell, the DNA needs to be introduced into the cell from its cell membrane. For this reason, the surface area is preferably as small as possible. For example, in a case of DNAs with the same sequence, a circular one is more preferable than a stranded one, and a supercoiled DNA resulting from twisting of a DNA is more preferable.

### <Proteins>

Proteins to be handled in the present embodiment include proteins and the like dissolved, dispersed, or dispersed in a state of being supported on a substrate in order to be introduced into the cell suspension. It suffices that their structure be a primary structure including an amino acid sequence and containing a polypeptide, a secondary structure such as α-helix or β-sheet, a tertiary structure including these secondary structures, and the quaternary structure of hemoglobin or the like, and higher-order structures corresponding to the purpose can be used. Specific examples include enzyme proteins such as amylase, structural proteins such as collagen and keratin, transport proteins such as albumin, storage proteins such as ferritin, and contractile proteins such as actin and myosin. The specific examples also include protective proteins such as globulin, modulated proteins such as calmodulin, as well as various membrane proteins, zinc-finger nuclease for genome editing, the Cas9 protein used in CRISPR/Cas9, and the like.

### <Labeling Substance>

A labeling substance to be handled in the present embodiment only needs to be such that, in a state where it is introduced in a cell, a label is recognizable from outside the cell. Such a labeling substance may be introduced to any of the nucleic acids or proteins mentioned above via chemical or physical modification. It suffices that the absorption wavelength or luminous wavelength of the labeling substance be different from that of the compound introduction-target cell and be recognizable. Also, it suffices that the labeling substance be present in a dissolved state, a dispersed state, or a dispersed state in a state of being supported on a substrate in order to be introduced into the cell suspension. Specific examples include stable isotopic substances such as deuterium, C13, and N15, radioactive substances, dyes, fluorescent dyes, pigments, fluorescent pigments, quantum dots, nanodiamonds, fullerenes, carbon nanosheets, carbon nanotubes, and the like.

### <Cell Types>

Cells to be handled in the present embodiment include adherent cells, suspension cells, spheroids {cell aggregates}, and the like. Specific examples thereof include cells of human cervical cancer cell lines, neurons, hepatocytes, fibroblasts, myoblasts, smooth muscle cells, cardiac muscle cell, skeletal muscle cells, stem cells, mesodermal stem cells, embryonic stem cells, glial cells, fetal stem cells, and hematopoietic stem cells. The specific examples also include mast cells, adipocytes, neural stem cells, and blood cells, and the like.

Other examples include microbial cells and plant cells. More specifically, these include prokaryotes such as Escherichia coli, Streptomyces, Bacillus subtilis, Streptococcus, and Staphylococcus, eukaryotic cells such as yeast and Aspergillus, insect cells such as Drosophila S2 and Spodoptera Sf9, and the like. Of these, eukaryotic cells are preferable cells. Preferably, the diameter of the cells (the average value of all cells) is such that a cell can be ejected from an ejection port and is, for example, 1 μm or more and 100 μm or less.

### <Cell Suspension>

The cell suspension has at least a compound and cells into which this compound is to be introduced. Further, in the present invention, the cell suspension is a liquid in which cells are dispersed. The cells in the cell suspension have only to be in a state in which the cells can be dispersed in the liquid by agitating, and may be precipitated in the liquid in a case where the cell suspension is kept in a stationary state. Incidentally, other components are preferably contained as appropriate so that the cells can survive during introduction processing and after it. These other components include salts, sugars, ribonucleotides, growth factor or hormone, pH buffer, surfactant, chelator, water-soluble organic solvent, proteins and amino acids, antibacterial agent, humectant, thickener, and the like.

### <Salts>

Examples of the salts to be handled in the present embodiment only need to be inorganic or organic salts for use in cell culture. Specifically, they include sodium chloride, potassium chloride, sodium citrate, and the like.

### <Sugars>

As examples of the sugars to be handled in the present embodiment, sugars as nutrient components for cells, sugars for adjusting the osmotic pressure, and the like can be used. Specifically, they include glucose, sucrose, fructose, and the like.

### <Ribonucleotides>

As examples of the ribonucleotides to be handled in the present embodiment, ribonucleotides for assisting cellular metabolism can be used. Specifically adenosine triphosphate, guanosine triphosphate, and the like.

### <Growth Factor or Hormone>

Examples of the growth factor or hormone to be handled in the present embodiment include human growth hormones. The examples also include growth hormones of other animals (such as bovine, porcine, and chicken growth factors), insulin, oxytocin, angiotensin, methionine enkephalin, and substance P. The examples also include ET-1, FGF, KGF, EGF, IGF PDGF, LHRH, GHRH, FSH, DDAVP, PTH, vasopressin, glucagon, somatostatin, and the like.

### <pH Buffer>

Examples of the pH buffer to be handled in the present embodiment include a citrate buffer solution, a phosphate buffer solution, a Tris buffer solution, or a HEPES buffer solution, and the like.

### <Surfactant>

Examples of the surfactant to be handled in the present embodiment include a water-soluble anionic surfactant, a water-soluble cationic surfactant, a water-soluble amphoteric surfactant, and a water-soluble nonionic surfactant, and one of these may be added or two or more may be added.

### <Chelator>

Specific examples of the chelator to be handled in the present embodiment include ethylenediaminetetraacetic acid (EDTA), glycol ether diamine tetraacetic acid (EGTA), and the like.

### <Water and Water-Soluble Organic Solvent>

The cell suspension to be handled in the present embodiment can use an aqueous liquid medium containing water or a mixture of water and a water-soluble organic solvent. The cell suspension can be obtained by adding the cells and the introduction-target compound to the aqueous liquid medium.

The solvent to be used in the present embodiment is not particularly limited, and examples thereof include water and saline. The examples also include a phosphate buffer solution (hereinafter PBS), a buffer solution of Tris or the like, and Dulbecco's Modified Eagle Medium (hereinafter D-MEM). The examples also include Iscove's Modified Dulbecco's Medium (hereinafter IMDM), Hanks' Balanced Salt Solutions (hereinafter HBSS), and the like. The examples also include Minimum Essential Medium-Eagle, Earle's Salts Base, with Non-Essential Amino Acid (hereinafter MEM-NEAA). The examples also include culture solutions for cell culture such as Roswell Park Memorial Institute Medium (RPMI) 1640 and the like. The examples also include commercially available buffers for electroporation, commercially available buffers for FACS analysis, and the like, as well as infusion solutions such as lactated Ringer's solution. It is particularly preferable that these solvents contain 50% water or more. Also, two or more of these solvents can be mixed and used. The water is preferably water deionized by ion exchange or the like and sterilized by heating with an autoclave or the like. Also, the content of water in the cell suspension medium is preferably 30% by mass or more and 99% by mass or less relative to the mass of the cell suspension.

The water-soluble organic solvent is not limited to a particular kind, and any publicly known organic solvent can be used according to the purpose. Specific examples include glycerin, polyethylene glycol, dimethyl sulfoxide, and the like. The content of the water-soluble organic solvent in the cell suspension is preferably 0.001% by mass or more and 50% by mass or less relative to the entire mass of the cell suspension.

### <Proteins and Amino Acids>

Examples of the proteins and amino acids to be handled in the present embodiment include serums such as fetal bovine serum (hereinafter FBS) and horse serum.

### <Antibacterial Agent>

Examples of the antibacterial agent to be handled in the present embodiment include antibiotics such as sodium azide and penicillin-streptomycin, and each can be used by adding it to the cell suspension. In particular, saline, PBS or a buffer solution of Tris or the like, a culture medium for cell culture such as D-MEM, IMDM, or HBSS, a commercially available buffer for FACS analysis, or the like, an infusion solution such as lactated Ringer's solution, or the like is preferably used for controlling the salinity, pH, or the like suitable for the cells.

### <Humectant>

Examples of the humectant to be handled in the present embodiment include polyalcohols such as glycerin, propylene glycol, butylene glycol, and sorbit. The examples also include mucopolysaccharides such as hyaluronic acid and chondroitin sulfate, hydrolyzed proteins soluble collagen, elastin, and keratin, and the like. One of these may be used alone or two or more may be mixed and used.

### <Thickener>

Examples to the thickeners to be handled in the present embodiment include starches such as an oxidatively modified starch, an enzymatically modified starch, a thermo-chemically modified starch, a cationic starch, an amphoteric starch, and an esterified starch. The examples also include cellulose derivatives such as carboxymethyl cellulose, hydroxyethyl cellulose, and ethyl cellulose, and natural or semisynthetic polymers such as casein, gelatin, or soybean protein. The examples also include completely or partially saponified water-soluble polymeric compounds. The water-soluble polymeric compounds include polyvinyl alcohol, acetoacetylated polyvinyl alcohol, and carboxy-modified polyvinyl alcohol. The examples also include polyvinyl alcohols such as olefin-modified polyvinyl alcohol and silyl-modified polyvinyl alcohol. At least one water-soluble polymeric compound can be selected from among these as appropriate and used. In the present embodiment, the viscosity of the liquid to be used is preferably 0.1 pascal-second (Pa·s) or more, and any of the above thickeners may be added as needed.

### <Method of Preparing Cell Suspension>

In a cell suspension in the present embodiment, cells cultured by adherent culture, suspension culture, or the like are separated into single cells or small cell aggregates via an action of an enzyme or the like and then, with a centrifuge or the like, only the cells are caused to settle by utilizing the difference in relative density. Thereafter, the supernatant medium excluding the cells is removed, and then the medium containing the introduction-target compound is added followed by agitation with a pipette or an agitator. As a result, a cell suspension is prepared.

The concentration of the cell suspension to be prepared may be adjusted according to the purpose and, is, in view of production efficiency, preferably 1,000 cells/mL or more and 100,000,000 cells/mL, or less and more preferably 100,000 cells/mL or more and 10,000,000 cells/mL or less.

Incidentally, it is preferable that, before the introduction of the cell suspension into the ejection head 101 to be used, the cell suspension be passed through a cell strainer having substantially the same diameter as the smallest diameter of the liquid supply flow channels in the ejection head. In this way, large cell aggregates can be excluded from the cell suspension.

### <Method of Culture Operation>

The cells processed can be cultured in an intended culture system. Specifically, in a case of animal cells, the cells are preferably cultured in an incubator in which is kept a saturated aqueous vapor at a culture temperature of 37 degrees Celsius and a carbon dioxide concentration of 5%. During the culture, it is preferable to check the cell's state of growth and replace the culture medium or perform passaging.

<Method of Checking Introduction of Compound into Cells>

The method of checking the introduction of the compound into the cells varies depending on the compound to be introduced. Thus, a suitable checking method may be used as appropriate. For example, in a case where a plasmid DNA that expresses GFP, which is a fluorescent protein, is introduced into the cells, a sample after the elapse of a certain time may be checked with a fluorescence microscope as to whether light is emitted from GFP. In this way, the amount to be introduced can be checked semiquantitatively. Also, the cultured cells may be separated into single cells by using an enzyme or the like and then the number of light-emitting cells may be counted by flow cytometry. In this way, the introduction can be checked quantitatively. Further, the cells can be dissociated and measured using a fluorescence spectrophotometer, a luminometer, or the like. Moreover, it is possible to use ELISA or immunostaining using antigen-antibody reaction. It is also possible to measure the introduced DNA and amplified DNA in the cells by using a real-time PCR apparatus or the like. In a case where the introduced compound is a labeling compound, an analysis can be made using an analysis unit for use in common chemical analyses.

### EXAMPLE

An example in the present embodiment will be described below. Note that the following example is a mere instance, and the present embodiment is not limited to this instance. “%” in some sentences are based on mass unless otherwise noted.

Firstly, the ejection head 101 was washed with a plenty of sterile water. Further, in a biological clean bench, the inside and outside of the ejection head 101 were cleaned and disinfected using an aqueous solution containing ethanol at a concentration of 70%. After removing an excess portion of the ethanol aqueous solution, the inside and outside of the ejection head 101 were washed using 15 mL of a 1× phosphate-buffered saline (1×PBS) (manufactured by Thermo Fisher Scientific K.K., pH=7.4). Further, 5 mL of 1×PBS was additionally introduced into the reservoir part 201 of the ejection head 101. In this state, the 1×PBS was sucked out from the ejection port surface of the ejection head in a communicating state by using an external aspirator connected to a sterilized tube. This operation was performed three times. As a result, the ejection head 101 sterilized was obtained.

The cell suspension and the introduction-target compound used in the compound introduction apparatus 100 in FIG. 1 were prepared as below.

### (Liquid Composition 1 Containing Introduction-Target Compound)

As described in Table 1 below, 500 μl of a trypan blue solution containing trypan blue as the introduction-target compound and 500 μl of Gene Pulser electroporation buffer (manufactured by Lanza K.K.) were mixed in a microtube to thereby obtain a liquid composition 1.

### (Liquid Composition 2 Containing Introduction-Target Compound)

As the introduction-target compound, a DNA solution containing DNA was prepared as below.

First, 0.2 mL of 0.5 mol/L-EDTA Solution (pH 8.0) (manufactured by NACALAI TESQUE, INC.) and 1 mL of 1 mol/L-Tris-HCl Buffer Solution (pH 8.0) (manufactured by NACALAI TESQUE, INC.) were mixed to thereby obtain a mixed liquid.

Next, 98.8 mL of sterilized pure water was added to this mixed liquid to thereby prepare a TE buffer (10 mM Tris 1 mM EDTA (pH 8)). Freeze-dried CMV-Fresno RFP (manufactured by ATUM, the number of base pairs=5.5 kbp) and the prepared TE buffer were mixed and agitated in a microtube, and the DNA was dissolved in that mixture. As a result, a DNA solution was obtained. Part of the obtained DNA solution was further diluted with the TE buffer and then filled into a quartz cell. Thereafter, with a DNA concentration measurement apparatus (GeneQuant 1300, manufactured by Biochrom), concentration identification was performed to thereby figure out the concentration of the DNA solution. The DNA concentration was 2.0 μg/μL.

As described in Table 1 below, 500 ml of the obtained DNA solution and 500 μl of Gene Pulser electroporation buffer (manufactured by Lonza K.K.) were mixed in a microtube to thereby obtain a liquid composition 2.

TABLE 1

| Liquid | | | | |
|---|---|---|---|---|
| Composition | Introduced Compound | | Base Liquid | |
| No. | Type | Amount μl | Type | Amount μl |
| Liquid | 0.4 w/v % | 500 | Gene Pulser | 500 |
| Composition | Trypan | | Electro- | |
| 1 | Blue Solution | | poration | |
| | (FUJIFILM | | Buffer | |
| | Wako | | | |
| | Pure Chemical | | | |
| | Corporation) | | | |
| Liquid | DNA Solution | 500 | Gene Pulser | 500 |
| Composition | | | Electro- | |
| 2 | | | poration | |
| | | | Buffer | |

The cell suspension was prepared through the following procedure.

RAW 264.7 purchased from the American Type Culture Collection, which was a cell line established from murine monocytic leukemia, was subjected to the following procedure so as to increase to 2,000,000 cells/mL.

The cell line, or RAW 264.7, was dispersed in 20 mL of a D-MEM culture medium containing 10% FBS, 1% penicillin-streptomycin, and 1% MEM-NEAA so as to increase to 2,000,000 cells/mL. Thereafter, groups of approximately 2,000,000 cells were seeded on a 100-mm polystyrene dish.

As the 10% FBS, one manufactured by Global Life Science Technologies Japan K.K. was used. As the 1% penicillin-streptomycin, one manufactured by Sigma-Aldrich Co. LLC. was used. As the 1% MEM-NEAA, one manufactured by Thermo Fisher Scientific K.K. was used. As the D-MEM, one manufactured by Thermo Fisher Scientific K.K. was used. As the 100-mm polystyrene dish, one manufactured by Corning Incorporated was used.

A dish having the D-MEM culture medium containing the seeded cells was incubated at 37° C. in the presence of 5% CO₂ to thereby amplify the cells. After two to four days, a state where the cells covered approximately 70% of the bottom surface of the dish was confirmed. Then, the supernatant culture medium was removed, followed by rinsing with PBS. The cells were detached from the dish by using PBS containing 0.25% trypsin and 1 mM EDTA (manufactured by Thermo Fisher Scientific K.K.). Thereafter, the cell suspension containing the RAW 264.7 cells was collected from the dish.

Inside a sterilized centrifuge tube, the above-mentioned D-MEM culture medium was added to the cell suspension collected from the dish such that the total amount would be 50 mL, followed by processing with a centrifuge (CF16RX II, manufactured by Hitachi Koki, Ltd.) set at a centrifugal force of 90 G at a temperature of 4 degrees Celsius for five minutes to thereby cause the cells to settle. The supernatant over the settled cell pellet was quietly removed, and then the above-mentioned D-MEM culture medium was added to the cell pellet. As a result, a cell suspension was obtained. This cell suspension was subjected to the above-described cell culture operation again twice.

In the third operation, a number of cells necessary to achieve a desired cell concentration were taken into another separate centrifuge tube. Then, after centrifugation, the above-mentioned liquid composition 1 was added instead of the above-mentioned D-MEM culture medium, followed by agitation with a micropipette. Further, the resultant liquid was passed through a cell strainer (manufactured by Corning Incorporated, mesh size=40 μm). As a result, a cell suspension 1 (2000000 cells/μl) to be used in the introduction of the compound into the cells was obtained. Also, a cell suspension 2 was obtained by a method similar to that for the cell suspension 1 except that the liquid composition 2 was used instead of the liquid composition 1.

The obtained cell suspensions 1 and 2 were each filled in a countess cell counting chamber slide (manufactured by Thermo Fisher Scientific K.K.) and an image was recorded using a phase-contrast microscope (manufactured by Olympus Corporation, model number: CKX41). Thereafter, by using the recorded image, the diameters of the longest portions of 1,000 cells were measured to derive the cell diameter. Note that the cell diameter derived was the cell diameter at 50% in a cell count-based cell diameter distribution obtained by the measurement.

### Example 1

In this example, the compound introduction apparatus 100 in FIG. 1 was used to introduce the compound into the cells as below.

First, 200 μL of the cell suspension 1 prepared by the above-described operations was introduced into the ejection head 101 by using a micropipetter. Thereafter, the ejection head 101 was conveyed to and brought into contact with the suction mechanism 104 and then the suction motor 105 was actuated, so that the cell suspension 1 was filled into the flow channels and the ejection ports 3 in the ejection head 101. After the filling, an introduction operation program was executed. As a result of executing the introduction operation program, the ejection head 101 was separated from the suction mechanism 104 and conveyed to above a glass bottom dish (manufactured by Iwaki) set in advance whose inner bottom surface was wetted by 100 μL of the above-described D-MEM culture medium. Thereafter, using all ejection ports 3, a liquid ejection operation for outputting a 100% processing duty image (specifically, a 1.5 cm×1.5 cm solid image) was performed to thereby eject the cell suspension 1 into the glass bottom dish. As a result, a sample was obtained. This liquid ejection operation was repeated 40 times, and continuous processability (“Possible Number of Successive Repetitions of Printing” in Table 2) was checked. Note that, with the compound introduction apparatus used in this example, “100%-processing duty” means to apply a single 23.0 n(nano)g droplet of the cell suspension to a 1/600 inch× 1/600 inch unit region at a resolution of 600 dpi×600 dpi. Incidentally, after the liquid ejection operations, the ventilation fan motor 210 was driven to actuate an air suction fan at the air suction port 109.

The sample thus obtained was covered with the glass bottom dish's top plate and taken out of the apparatus, and 2.5 mL of the above-described D-MEM culture medium warmed up to 37° C. was quietly poured using a micropipette. Then, using a phase-contrast microscope (manufactured by Olympus Corporation, model number: CKX41), the cells were observed, and the processing rate was derived.

### <<Evaluation of Processing Rate>>

The processing rate of the cells into which the compound was introduced using the cell suspension 1 as described above was measured. Specifically, the cells were observed using the phase-contrast microscope with a 10× object lens in a bright-field mode to count the number of cells colored in purple with the trypan blue and the number of all cells. Moreover, the processing rate was calculated by following an equation Processing Rate=Number of Purple Cells/Number of All Cells×100, and the calculated processing rate was evaluated based on the following five criteria. The result of this evaluation is described in Table 2. Note that in this example, rank C and below are defined as unacceptable levels in terms of production efficiency.

AA: Processing rate of 50% and more

A: Processing rate of 40% or more and less than 50%

B: Processing rate of 20% or more and less than 40%

C: Processing rate of 10% or more and less than 20%

D: Processing rate of less than 10%

### Examples 2 and 5 and Comparative Examples 1 and 2

As described in Table 2, samples in Examples 2 and 5 and Comparative Examples 1 and 2 were prepared by performing similar operations to Example 1 except that the diameter of the ejection ports, the height and width of the liquid supply flow channels, and the nozzle thickness were changed. The processing rates of the samples obtained were each evaluated by a similar method to Example 1. The result of this evaluation is described in Table 2. Note that, in Comparative Example 1, a liquid ejection operation could not be repeated 40 times. For this reason, “Possible Number of Successive Repetitions of Printing” in Table 2 is “C”, and the processing rate was not evaluated either.

### Example 3

A sample was prepared by performing similar operations to Example 1 except that the cell suspension 2 was used instead of the cell suspension 1. Then, the sample obtained was incubated at 37° C. in the presence of 5% CO₂, so that the cells were amplified. 24 hours later, the gene expression was evaluated using a fluorescence microscope to determine whether DNA was introduced into the cells.

### <<Evaluation of Gene Expression>>

The CMV-Fresno RFP used in this example contains a gene that generates a protein which emits red fluorescence in a case where it is introduced into a cell. Thus, expression of this fluorescent protein was utilized to evaluate the DNA introduction.

Specifically, from the sample after the 24-hour incubation, the culture medium was removed, followed rinsing with 1×PBS and addition of 2 mL of 1×PBS. After the culture medium was replaced with 1×PBS, the sample was observed using a fluorescence microscope (manufactured by Keyence Corporation, model number: BZ-8000) with a 10× object lens in a bright-field mode and a fluorescence mode (TRITC: excitation=540±12.5 nm, fluorescence=605±27.5 nm, cut=565 nm). Then, the number of fluorescence-emitting cells and the number of all cells were counted, and the introduction rate was calculated by following an equation Introduction Rate=(Number of Fluorescence-Emitting Cells/Number of All Cells)×100. The calculated introduction rate was evaluated based on the following criteria. The result of this evaluation is described in Table 2.

Expressed: Introduction rate of 1% or more

Not Expressed: Introduction rate of less than 1%

### Examples 4 and 6

As described in Table 2, samples in Examples 4 and 6 were prepared by performing similar operations to Example 3 except that the diameter of the ejection ports, the height and width of the liquid supply flow channels, and the nozzle thickness were changed. The cells in the samples obtained were amplified by a similar method to Example 3, and the gene expression was evaluated similarly to Example 3. The result of this evaluation is described in Table 2.

### Comparative Examples 3 and 4

Comparative Examples 3 and 4 will be described below as comparative examples of Examples 1 and 3. Specifically, in Comparative Examples 3 and 4, in order to check the effect of the bubble generation, the heat generation elements were not caused to generate bubbles, and the cell suspension was taken out and added into a glass bottom dish by external suction through the ejection ports instead of the ejection operation. Besides the above, similar operations to Examples 1 and 3 were performed to prepare samples with no bubble generation process. By using the samples prepared, the processing rate was evaluated for Comparative example 3, and the gene expression was evaluated for Comparative Example 4. The results of these evaluations are described in Table 2.

TABLE 2

| | | | | | | | Comp. | Comp. | Comp. | Comp. |
|---|---|---|---|---|---|---|---|---|---|---|
| | Ex. 1 | Ex. 2 | Ex. 3 | Ex. 4 | Ex. 5 | Ex. 6 | Ex. 1 | Ex. 2 | Ex. 3 | Ex. 4 |
| Ejection Port | 15 | 23 | 15 | 23 | 15 | 15 | 10 | 25 | 15 | 15 |
| Diameter (μm) | | | | | | | | | | |
| Supply Flow | 14 | 20 | 14 | 20 | 20 | 20 | 14 | 20 | 14 | 14 |
| Channel Height | | | | | | | | | | |
| (μm) | | | | | | | | | | |
| Supply Flow | 20 | 25 | 20 | 25 | 20 | 20 | 20 | 25 | 20 | 20 |
| Channel Width | | | | | | | | | | |
| (μm) | | | | | | | | | | |
| Nozzle Thickness | 25 | 72 | 25 | 72 | 25 | 25 | 25 | 72 | 25 | 25 |
| (μm) | | | | | | | | | | |
| Cell Diameter | 12 | 12 | 12 | 12 | 12 | 12 | 12 | 12 | 12 | 12 |
| (μm) | | | | | | | | | | |
| Ejection Port | 1.3 | 1.9 | 1.3 | 1.9 | 1.3 | 1.3 | 0.8 | 2.1 | 1.3 | 1.3 |
| Diameter/Cell | | | | | | | | | | |
| Diameter | | | | | | | | | | |
| Supply Flow | 1.2 | 1.7 | 1.2 | 1.7 | 1.7 | 1.7 | 1.2 | 1.7 | 1.2 | 1.2 |
| Channel Height/ | | | | | | | | | | |
| Cell Diameter | | | | | | | | | | |
| Supply Flow | 1.7 | 2.1 | 1.7 | 2.1 | 1.7 | 1.7 | 1.7 | 2.1 | 1.7 | 1.7 |
| Channel Width/ | | | | | | | | | | |
| Cell Diameter | | | | | | | | | | |
| Ejection Port | 1.1 | 1.2 | 1.1 | 1.2 | 0.8 | 0.8 | 0.7 | 1.3 | 1.1 | 1.1 |
| Diameter/Supply | | | | | | | | | | |
| Flow Channel | | | | | | | | | | |
| Height | | | | | | | | | | |
| Bubble | Generated | Generated | Generated | Generated | Generated | Generated | Generated | Generated | Not | Not |
| Generation | | | | | | | | | Generated | Generated |
| Liquid | Liquid | Liquid | Liquid | Liquid | Liquid | Liquid | Liquid | Liquid | Liquid | Liquid |
| Composition | Composi- | Composi- | Composi- | Composi- | Composi- | Composi- | Composi- | Composi- | Composi- | Composi- |
| | tion 1 | tion 1 | tion 2 | tion 2 | tion 1 | tion 2 | tion 1 | tion 1 | tion 1 | tion 2 |
| Processing Rate | AA | AA | | | A | | | C | D | |
| Possible Number | 40 | 40 | 40 | 40 | 40 | 40 | C | 40 | | |
| of Successive | | | | | | | | | | |
| Repetitions of | | | | | | | | | | |
| Printing | | | | | | | | | | |
| Gene Expression | | | Expressed | Expressed | | Expressed | | | | Not |
| | | | | | | | | | | Expressed |

According to one embodiment of the present invention, it is possible to achieve both efficient introduction of a compound into a cell and efficient generation of a cell whose properties are modified by the introduction.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-044714, filed Mar. 18, 2021, and No. 2022-037194, filed Mar. 10, 2022 which are hereby incorporated by reference wherein in their entirety.

What is claimed is:

1. A compound introduction apparatus for introducing a compound into a cell, comprising:
a supply flow channel for supplying a cell suspension containing the cell and the compound;
an introduction unit configured to introduce the compound into the cell; and
an ejection port for ejecting the cell suspension containing the cell in which the compound has been introduced, wherein
a height of the supply flow channel relative to a flow direction of the cell suspension flowing through the supply flow channel is more than 1.0 times a diameter of the cell and less than 2.0 times the diameter of the cell, and a width of the supply flow channel relative to the flow direction is more than 1.0 times the diameter of the cell and less than 2.0 times the diameter of the cell, and
a diameter of the ejection port is more than 1.0 times the diameter of the cell and less than 2.0 times the diameter of the cell.

2. The compound introduction apparatus according to claim 1, wherein the introduction unit is an energy generation element that generates energy in a height direction of the supply flow channel.

3. The compound introduction apparatus according to claim 1, wherein the introduction unit is a heat generation element.

4. The compound introduction apparatus according to claim 3, further comprising a processing chamber in which the heat generation element is arranged.

5. The compound introduction apparatus according to claim 4, wherein the ejection port is arranged directly below the heat generation element and the processing chamber.

6. The compound introduction apparatus according to claim 4, wherein in a case where the heat generation element generates heat, a stress resulting from bubble generation caused by the heat generation is applied to the cell suspension inside the processing chamber.

7. The compound introduction apparatus according to claim 6, wherein the stress is exerted for 1 μs or more and 10 μs or less and consequently a droplet of the cell suspension is discharged from the ejection port at a speed of 0.5 m/s or more and 30 m/s or less.

8. The compound introduction apparatus according to claim 3, wherein a time for which the heat generation element generates heat is 0.1 μs or more and 5 us or less.

9. The compound introduction apparatus according to claim 1, wherein the diameter of the ejection port is larger than the height of the supply flow channel.

10. The compound introduction apparatus according to claim 1, wherein the diameter of the cell is 1 μm or more and 100 μm or less.

11. The compound introduction apparatus according to claim 1, wherein the height and the width of the supply flow channel are each more than 1 μm and less than 200 μm.

12. The compound introduction apparatus according to claim 1, wherein the cell is a eukaryotic cell.

13. The compound introduction apparatus according to claim 1, wherein the height of the supply flow channel means a length thereof in a direction of gravity.

14. A compound introduction method of introducing a compound into a cell, comprising:
supplying a cell suspension containing the cell and the compound through a supply flow channel;
introducing the compound into the cell; and
ejecting, from an ejection port, the cell suspension containing the cell in which the compound has been introduced, wherein
a height of the supply flow channel relative to a flow direction of the cell suspension flowing through the supply flow channel is more than 1.0 times a diameter of the cell and less than 2.0 times the diameter of the cell, and a width of the supply flow channel relative to the flow direction is more than 1.0 times the diameter of the cell and less than 2.0 times the diameter of the cell, and
a diameter of the ejection port is more than 1.0 times the diameter of the cell and less than 2.0 times the diameter of the cell.
